(19) United States
(12) Patent Application Publication
(10) Pub. No.: US 2012/0238770 A1
(43) Pub. Date: Sep. 20, 2012

(54) GLYCIDYL ESTER REDUCTION IN OIL

(75) Inventors: Scott Bloomer, Decatur, IL (US); Phil Hogan, Hamburg (DE); John Inmok Lee, Decatur, IL (US); Mark Matlock, Decatur, IL (US); Lisa M. Pfalzgraf, Decatur, IL (US); Leif Solheim, Decatur, IL (US); Lori E. Wicklund, Argenta, IL (US)

(73) Assignee: ARCHER DANIELS MIDLAND COMPANY, Decatur, IL (US)

(21) Appl. No.: 13/512,626

(22) PCT Filed: Dec. 3, 2010
(86) PCT No.: PCT/US2010/058819

§ 371 Date: May 30, 2012

Related U.S. Application Data
(60) Provisional application No. 61/266,780, filed on Dec. 4, 2009.
(60) Provisional application No. 61/363,300, filed on Jul. 12, 2010.

(51) Int. Cl. C07C 57/03; C07C 51/42; C12P 7/64

(52) U.S. Cl. 554/223; 435/134; 554/191; 554/175

(57) ABSTRACT

Vegetable oils having a low level of glycidol esters are disclosed. Methods for reduction of the content of glycidol esters in edible oils are also disclosed.

This application claims priority to U.S. Provisional Patent Application No. 61/266,780, filed Dec. 4, 2009 and to U.S. Provisional Patent Application No. 61/363,300, filed Jul. 12, 2010.

### TECHNICAL FIELD

Glycidol esters have been found in vegetable oils. During digestion of such vegetable oils, glycidol esters may release glycidol, a known carcinogen. The present invention provides for vegetable oils having a low level of glycidol esters, as well as methods of removing glycidol esters from oil.

One non-limiting aspect of the present disclosure is directed to a method of removing glycidyl esters from oil, wherein the method includes contacting the oil with an adsorbent, and subsequently steam refining the oil. In certain non-limiting embodiments of the method, steam refining the oil includes at least one of deodorization and physical refining. Also, in certain non-limiting embodiments of the method the adsorbent comprises at least one material selected from magnesium silicate, silica gel, and bleaching clay.

An additional non-limiting aspect of the present disclosure is directed to a method of removing glycidyl esters from oil, wherein the method includes contacting the oil with an enzyme, and subsequently steam distilling the oil. In certain non-limiting embodiments of the method, contacting the oil with an enzyme includes at least one reaction selected from hydrolysis, esterification, transesterification, acidolysis, interesterification, and alcoholysis.

Another non-limiting aspect of the present disclosure is directed to a method of removing glycidyl esters from oil, wherein the method includes deodorizing the oil at a temperature no greater than 240 degrees C. According to certain non-limiting embodiments of the method, the oil includes at least one oil selected from palm oil, palm fractions, palm olein, palm stearin, corn oil, soybean oil, esterified oil, interesterified oil, chemically interesterified oil, and lipase-contacted oil.

Yet another non-limiting aspect of the present disclosure is directed to a method of removing glycidyl esters from oil, wherein the method includes deodorizing the oil with at least one sparge selected from ethanol sparge, carbon dioxide sparge, and nitrogen sparge.

A further non-limiting aspect of the present disclosure is directed to a method of removing glycidyl esters from oil, wherein the method includes contacting the oil with a solution including an acid. In certain non-limiting embodiments of the method, the solution comprises phosphoric acid. Also, in certain non-limiting embodiments of the method, contacting the oil with the solution includes shear mixing the oil and the solution.

Yet a further non-limiting aspect of the present disclosure is directed to a method of removing glycidyl esters from bleached oil, wherein the method includes rebleaching the oil. In certain non-limiting embodiments of the method, the bleached oil includes at least one of refined bleached oil, refined bleached deodorized oil, and chemically interesterified oil. Also, in certain non-limiting embodiments of the method, the method includes deodorizing the oil subsequent to rebleaching the oil.

A still further non-limiting aspect of the present disclosure is directed to a method of removing glycidyl esters from oil, wherein the method includes contacting the oil with an adsorbent.

Another non-limiting aspect of the present disclosure is directed to a composition including physically refined palm oil having a level of glycidyl esters less than 0.1 ppm as determined by liquid chromatography time-of-flight mass spectroscopy.

An additional non-limiting aspect of the present disclosure is directed to a composition including palm olein having a level of glycidyl esters less than 0.1 ppm as determined by liquid chromatography time-of-flight mass spectroscopy.

A further non-limiting aspect of the present disclosure is directed to a composition including physically refined palm olein having a level of glycidyl esters less than 0.3 ppm as determined by liquid chromatography time-of-flight mass spectroscopy.

Yet a further non-limiting aspect of the present disclosure is directed to a composition including a rebleached, redeodorized oil, wherein the oil includes: a level of glycidyl esters less than 0.1 ppm as determined by liquid chromatography time-of-flight mass spectroscopy; a Lovibond red color value no greater than 2.0; a Lovibond yellow color value no greater than 20.0; and a free fatty acid content of less than 0.1%. In certain non-limiting embodiments of the composition, the rebleached, redeodorized oil includes flavor that passes the American Oil Chemists' Society method Cg-2-83.

Still a further non-limiting aspect of the present disclosure is directed to a composition including a rebleached, steam distilled palm oil, wherein the oil includes: a level of glycidyl esters below 0.2 ppm as determined by the liquid chromatography time-of-flight mass spectroscopy method; a Lovibond red color value no greater than 3.0; and less than 0.1% free fatty acids.

Yet another non-limiting aspect of the present disclosure is directed to a composition including a rebleached, steam distilled palm stearin, the palm stearin comprising: a level of glycidyl esters below 0.2 ppm as determined by the liquid chromatography time-of-flight mass spectroscopy method; a Lovibond red color value of 4.0 or less; and less than 0.1% free fatty acids.

A further non-limiting aspect of the present disclosure is directed to a composition including a bleached lipase-contacted oil including a level of glycidyl esters less than 1.0 ppm as determined by liquid chromatography time-of-flight mass spectroscopy. In certain non-limiting embodiments of the composition, the bleached lipase-contacted oil is deodorized.

Yet a further non-limiting aspect of the present disclosure is directed to a composition comprising a steam refined esterified oil including a level of glycidyl esters less than 1.0 ppm as determined by liquid chromatography time-of-flight mass spectroscopy.

Yet another non-limiting aspect of the present disclosure is directed to a composition including a rebleached soybean oil, the soybean oil comprising a level of glycidyl esters below 0.2 ppm as determined by the liquid chromatography time-of-flight mass spectroscopy method.

Yet a further non-limiting aspect of the present disclosure is directed to a method of removing glycidyl esters from bleached oil, wherein the method includes mixing water into the oil and rebleaching the oil. In certain non-limiting embodiments of the method, the bleached oil includes at least one of refined bleached oil, refined bleached deodorized oil, and chemically interesterified oil. Also, in certain non-limiting embodiments of the method, the method includes deodorizing the oil subsequent to rebleaching the oil.

Another non-limiting aspect of the present disclosure is directed to a method of converting glycidyl esters in oil into monoacylglycerols, wherein the method includes mixing water into the oil and rebleaching the oil. In certain non-limiting embodiments of the method, the bleached oil includes at least one of refined bleached oil, refined bleached deodorized oil, and chemically interesterified oil. Also, in certain non-limiting embodiments of the method, the method includes deodorizing the oil subsequent to rebleaching the oil.

As used herein, “deodorization” means distillation of alkali refined oil to remove impurities. Exemplary oils include but are not limited to soybean oil, canola oil, corn oil, sunflower oil, and safflower oil.

As used herein, “alkali refining” or “chemical refining” means removing free fatty acids from oil by contacting with a solution of alkali and removal of most of the resulting fatty acid soaps from the bulk of triacylglycerols. Alkali refined oil is often, but not always, subsequently deodorized.

As used herein, “physical refining” means high temperature distillation of oil under conditions which remove most free fatty acids while keeping the bulk of triacylglycerols intact.

As used herein, “steam refining” and “steam distillation” mean physical refining and/or deodorization.

As used herein, “hydrolysis” means the reaction of an ester with water, producing a free acid and an alcohol.

As used herein, “esterification” or “ester synthesis” means the reaction of an alcohol with an acid, especially a free fatty acid, leading to formation of an ester. During the esterification reactions described in this application, free fatty acids present in starting materials may react with polyhydric alcohol, such as glycerol or monoacylglycerols, or with monohydric alcohols, such as diacylglycerols.

As used herein, “acidolysis” means a reaction in which a free acid reacts with an ester, replacing the acid bound to the ester and forming a new ester molecule.

As used herein, “transesterification” means the reaction in which an ester is converted into another ester, for example by exchange of an ester-bound fatty acid from a first alcohol group to a second alcohol group.

As used herein, “alcoholysis” means a reaction in which a free alcohol reacts with an ester, replacing the alcohol bound to the ester and forming a new ester molecule.

As used herein, “interesterification” reactions mean the following reactions: acidolysis, transesterification, and alcoholysis.

As used herein, “lipase contacted,” “lipase-catalyzed reactions,” “contacting an oil with and enzyme,” and “incubating an oil with an enzyme” each mean one or more of the following reactions: hydrolysis, esterification, transesterification, acidolysis, interesterification, and alcoholysis.

As used herein, “acylglycerols” means glycerol esters commonly found in oil, such as monoacylglycerols, diacylglycerols, and triacylglycerols. As used herein, the term “partial glycerides” means glycerol esters having one or two free hydroxyl groups, such as monoacylglycerols and diacylglycerols.

As used herein, “palm fraction” means a component of palm oil obtained from fractionation of palm oil.

As used herein, “palm olein” means a palm fraction enriched in palm oil components having a lower melting point than either the unfractionated palm oil or palm stearin, or that is predominantly liquid oil at room temperature.

As used herein, “palm stearin” means a palm fraction enriched in palm oil components having a higher melting point than either the unfractionated palm oil or palm olein, or is predominantly solid oil at room temperature.

As used herein, “sparge” means the introduction of a gas phase into a liquid phase.

As used herein, “chemical interesterification” means the rearrangement of fatty acids in an oil catalyzed with chemical (non-biological) catalysts, such as, for example, sodium methoxide.

Given the inaccuracy of available, indirect methods of determining the level of glycidyl esters in oil, a direct method of determining the level of glycidyl esters in oil was developed. Existing, indirect methods of quantification of glycidyl esters rely on a chemical conversion of glycidyl esters with sodium methoxide to monochloropropanediol, which is the compound actually measured. However, this incorporates the incorrect assumption that glycidyl esters are the only species capable of being converted into the compounds which are actually measured. This indirect method is therefore prone to reporting incorrect levels of monochloropropanediol esters and glycidyl esters.

A new, more accurate method, which is described below and shall be referred to herein as “liquid chromatography time-of-flight mass spectroscopy” or “LC-TOFMS”, was used to determine the levels of glycidyl esters recited herein. Samples were prepared by dilution with mobile phase and separated by liquid chromatography. Detection was carried out using time-of-flight mass spectrometry. Samples were run daily to verify accurate identification and quantification.

MCPD fatty acid esters and glycidyl fatty acid esters were determined in vegetable oils by high performance liquid chromatography (HPLC) coupled to time-of-flight mass spectroscopy (TOFMS). Samples were diluted and injected without prior chemical modification and separated by reversed phase HPLC. Electrospray ionization was utilized, enhanced by the inclusion of a constant level of trace sodium salts in the chromatography. Variations in the level of sodium may lead to aberrant results, so ensuring a constant level of sodium is important. Analytes were detected as [M+Na(+)] ions. For HPLC separation, an Agilent 1200 series™ HPLC was used. The effluent was analyzed with Agilent 6210™ TOFMS using a Phenomenex Luna™ 3 micron C18 column (100 angstrom pore size, 50 mm×3.0 mm column). A two-solvent gradient was applied according to Table 2.

TABLE 2
HPLC gradient conditions

| Solvent A | 90% methanol:10% acetonitrile with 0.026 mM |
|---|---|
| | sodium acetate |
| Solvent B | 80% methylene chloride:10% methanol:10% |
| | acetonitrile with 0.026 mM sodium acetate |
| Flow Rate | 0.25 ml/min |
| Run Time | % Solvent B |
| 0 min | 0 |
| 15 min | 65 |
| 16 min | 100 |
| 20 min | 100 |

Standards were used to verify the identity and quantities of analytes detected. Several standards were obtained commercially as indicated in Table 3. Several standards were unavailable commercially and were synthesized in the laboratories of Archer Daniels Midland Company in Decatur, Ill. as also listed in Table 3.

TABLE 3
Standards for analysis

| | |
|---|---|
| 3-MCPD Monopalmitate | Toronto Research |
| 3-MCPD Monostearate | Toronto Research |
| 3-MCPD Dipalmitate | Toronto Research |
| Glycidyl Stearate | TCI America |
| Glycidyl Palmitate | Synthesized |
| Glycidyl Oleate | Synthesized |
| 3-MCPD Diolein | Synthesized |
| d5-3-MCPD Diolein | Synthesized |
| 3-MCPD Dilinolein | Synthesized |
| Mixed 3-MCPD C16-C18 Fatty Acid | Synthesized |
| Monoesters | |
| Mixed 3-MCPD C16-C18 Fatty Acid | Synthesized |
| Diesters | |
| Mixed Glycidyl C16-C18 Fatty Acid Esters | Synthesized |

Analyte names, retention times, molecular formula, and ions detected are given in Table 4.

TABLE 4
Analyte names, retention times, molecular formula, and ions detected by mass to charge ratio.

| | | | |
|---|---|---|---|
| | | | Mass/ |
| | | | charge ratio |
| | | | m/z Ion |
| | Retention | | Detected |
| Compound | Time (min.) | Formula | [M + Na(+)] |

Glycidol esters

| | | | |
|---|---|---|---|
| Palmitic Acid Glycidol Ester | 2.0 | C19H36O3 | 335.25622 |
| Stearic Acid Glycidol Ester | 2.3 | C21H40O3 | 363.28752 |
| Oleic Acid Glycidol Ester | 2.0 | C21H38O3 | 361.27187 |
| Linoleic Acid Glycidol Ester | 1.8 | C21H36O3 | 359.25622 |
| Linolenic Acid Glycidol Ester | 1.4 | C21H34O3 | 357.24057 |

MCPD monoesters

| | | | |
|---|---|---|---|
| Palmitic Acid MCPD monoester | 1.8 | C19H37CIO3 | 371.23289 |
| Stearic Acid MCPD monoester | 2.1 | C21H41CIO3 | 399.26419 |
| Oleic Acid MCPD monoester | 1.7 | C21H39CIO3 | 397.24854 |
| Linoleic Acid MCPD monoester | 1.7 | C21H37CIO3 | 395.23289 |
| Linolenic Acid MCPD monoester | 1.6 | C21H35CIO3 | 393.21724 |

MCPD diesters

| | | | |
|---|---|---|---|
| Palmitic Acid-Oleic Acid-MCPD diester | 8.8 | C37H69CIO4 | 635.47821 |
| di-Palmitic Acid MCPD Diester | 8.8 | C35H67CIO4 | 609.46256 |
| di-Oleic Acid MCPD diester | 9.3 | C39H71CIO4 | 661.49386 |
| Palmitic Acid-Linoleic Acid MCPD diester | 6.6 | C37H67CIO4 | 633.46256 |
| Oleic Acid-Linoleic Acid MCPD diester | 7.1 | C39H69CIO4 | 659.47821 |
| Palmitic Acid-Stearic Acid MCPD diester | 11.4 | C37H71CIO4 | 637.49386 |
| Oleic Acid-Stearic Acid MCPD Diester | 11.6 | C39H73CIO4 | 663.50951 |
| di-Linoleic Acid MCPD diester | 5.7 | C39H67CIO4 | 657.46256 |
| Linoleic Acid-Stearic Acid MCPD diester | 10.6 | C39H71CIO4 | 661.49386 |
| di-Stearic Acid MCPD diester | 14.0 | C39H75CIO4 | 665.52516 |
| di-Linolenic Acid MCPD diester | 3.9 | C39H63CIO4 | 653.43126 |
| Oleic Acid-Linolenic Acid MCPD diester | 5.1 | C39H67CIO4 | 657.46256 |
| Linoleic Acid-Linolenic Acid MCPD diester | 4.6 | C39H65CIO4 | 655.44626 |
| Palmitic Acid-Linolenic Acid MCPD diester | 5.4 | C37H65CIO4 | 631.44691 |
| Stearic Acid-Linolenic Acid MCPD diester | 9.7 | C39H69CIO4 | 659.47821 |

Internal Standard

| | | | |
|---|---|---|---|
| d5-MCPD Di-Oleic Acid Ester | 9.5 | C39H66D5CIO4 | 666.52524 |

Mass Reference Ions

| | | |
|---|---|---|
| Monoheptadecanoin | C20H40O4 | 367.28243 |
| Dinonadecanoin | C41H80O5 | 675.59035 |

Standards which were not commercially available were synthesized as follows:

Deuterated 3-MCPD diesters of oleic acid were synthesized as follows: oleic acid (30.7 grams, 99%+, Nu Chek Prep, Inc., Elysian, Minn.) and 5.07 g deuterated 3-MCPD (±-3-chloro-1,2-propane-d₅-diol, 98 atom % D, C/D/N Isopotes Inc, Pointe-Claire, Quebec, Canada) were reacted with 3.1g Novozym 435 immobilized lipase (Novozymes, Bagsvaerd, Denmark) at 45 C, under 5 mmHg vacuum, with vigorous agitation (450 rpm) for 70 hrs. There was 25% excess oleic acid on molar basis. TLC analysis indicated that almost all monoesters were converted to diesters after 70 hrs. After cooling to room temperature, 150 ml hexane was added to the reaction mixture and the reaction mixture was filtered through #40 filter paper (Whatman Inc., Florham Park, N.J.) to recover the enzyme granules. The hexane/reaction mixture solution was washed with caustic solution in a 500-ml separatory funnel to remove excess free fatty acids. 18 ml of 9.5 wt/v % NaOH solution was added to the separatory funnel and was shaken for 3 min for neutralization. After removal of lower soap phase, the upper phase was washed several times with 100 ml warm water until pH of the wash water became neutral. Hexane was evaporated in a rotary evaporator then by mechanical vacuum pump to completely remove residual hexane and moisture. After hexane removal, 20.6 g material was recovered. The finished material had less than 0.1% free fatty acid, by titration, and was expected to have 95% deuterated 3-MCPD diesters of oleic acid. Deuterated 3-MCPD diesters of Linoleic acid were prepared the same way using linoleic acid (99%+, Nu Chek Prep, Inc., Elysian, Minn.).

Deuterated 3-MCPD monoesters of oleic acid were prepared substantially as the Deuterated 3-MCPD diesters of oleic acid except the reaction time was shortened to 45 minutes. An emulsion formed, from which 1 gram deuterated 3-MCPD monoester of oleic acid containing 9.6% free fatty acid was recovered.

Glycidol palmitate was prepared as follows: a 250 mL 3 neck round bottom flask equipped with overhead stirrer, Dean-Stark trap and condenser was charged with 10 g methyl palmitate (99%+, Nu Chek Prep, Inc., Elysian, Minn.), 13.7 g glycidol (Sigma-Aldrich, St. Louis, Mo.) and 1 g Novozymes 435 immobilized lipase. The reaction mixture was heated to 70° C. using an oil bath and purged with nitrogen to remove any methanol formed during the reaction. The progress of the reaction was monitored by TLC (80:20 (v/v) hexanes:ethyl acetate). The reaction was stopped after 24 h. The reaction mixture was diluted with ethyl acetate and filtered to remove the immobilized enzyme. The solvent and excess glycidol was removed in vacuo to give a colorless oil that solidified upon cooling (13 g) into a crude product. Crude product (5 grams) was purified using column chromatography (0-20% ethyl acetate: hexanes (v/v)). Methyl palmitate eluted with hexanes. The product glycidyl palmitate eluted in 5-10% ethyl acetate: hexanes (v/v). Fraction containing the product were pooled and concentrate in vacuo to give a while solid (2 g) TLC plates were visualized by spraying with Hanessian stain followed by heating at 110° C. for 15 min.

Glycidol oleate was prepared as glycidol palmitate except that 10 grams of methyl oleate (99%+, Nu Chek Prep, Inc., Elysian, Minn.) and 13.1 grams of glycidol were used.

Detection by LC-TOFMS was carried out by mass spectrometry using ESI Source; Gas Temp.—300° C.; Drying Gas—5 L/min.; Nebulizer Pressure—50 psi. The mass spectrometer parameters were: MS Mass Range—300 to 700 m/z; Polarity—Positive; Instrument Mode—2 GHz; Data Storage—Centroid and Profile. Standards were included in sample sets each day of analysis. Quantities of glycidyl esters were reported in ppm. LC-TOFMS was able to detect the presence of each glycidyl ester at concentrations as low as 0.1 ppm. In each set of samples, if no glycidyl esters were detected, a limit of detection was estimated for that sample. Because the number of components and the ratio of the components is not uniform from sample to sample, the limit of detection achieved is not always identical. Both instrument conditions (how recently it was cleaned and tuned) and the type of sample being run affect the limit of detection that is achieved. The actual limit of detection achieved is reported for each Example below.

In addition to determination of glycidyl ester levels using LC-TOFMS, color and flavor were also determined in some samples as described below. Lovibond color values of vegetable oils were determined according to AOCS official method Cc 13b-45, in which oil color is determined by comparison with glasses of known color characteristics in a colorimeter. The free fatty acid content of vegetable oils was determined according to AOCS official method Ca 5a-40, in which free fatty acids are determined by titration and reported as percent oleic acid.

The flavor of vegetable oils was determined substantially according to A.O.C.S method Cg 2-83 (Panel Evaluation of Vegetable Oils) by two experienced oil tasters. About 15 ml oil was put into a 30 ml PET container and heated to ˜50° C. in a microwave oven, before tasting. Overall flavor quality score was rated on a scale of 1 to 10, with 10 being excellent. A sample did not pass unless the score was 7 or greater. All AOCS methods are from 6th edition of the “Official Methods and Recommended Practices of the AOCS,” Urbana, Ill.

### BRIEF DESCRIPTION OF FIGURE IN THE DRAWING

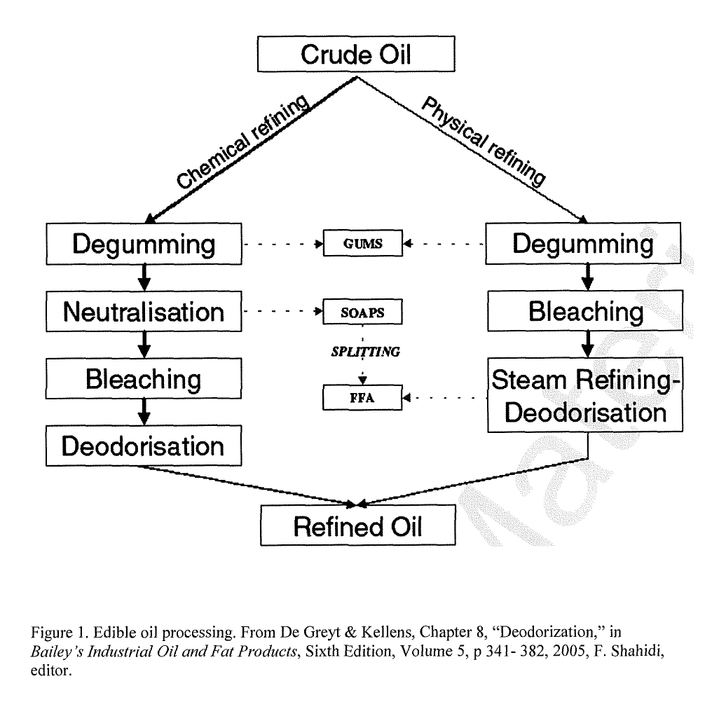

Reference is made to FIG. 1, which depicts edible oil processing and is taken from “Edible oil processing,” De Greyt & Kellens, Chapter 8, “Deodorization,” in Bailey's Industrial Oil and Fat Products, Sixth Edition, Volume 5, p 341-382, 2005, F. Shahidi, editor.

### EXAMPLES

The following examples illustrate methods for removing glycidyl esters from oil, and compositions of oils containing low levels of glycidyl esters, according to the present invention. The following examples are illustrative only and are not intended to limit the scope of the invention as defined by the appended claims.

### Example 1A

In a control experiment, bleached palm oil (Archer Daniels Midland (ADM) Hamburg, Germany) containing 0.8 ppm glycidyl esters was steam refined by physical refining at 260° C. for 30 minutes with 3% steam and 3 mm Hg vacuum substantially as follows: palm oil was charged into a 1-liter round-bottom glass distillation vessel fitted with a sparge tube, one opening of which was below the top of the oil level. The other opening of the sparge tube was connected to a vessel containing deionized water. The sparge tube was set to provide a total content of sparge steam of the desired percentage by weight of oil of steam throughout the deodorization process by drawing water into the oil due to the vacuum applied to the vessel headspace. The vessel was also fitted with a condenser through an insulated adapter. A vacuum line was fitted to the vessel headspace through the condenser, with a cold trap located between the condenser and the vacuum source. Vacuum (3 mm Hg) was applied and the oil was heated to 260° C. at a rate of 10° C./minute. This temperature was held for 30 minutes. A heat lamp was applied to the vessel containing deionized water to generate steam; the vacuum drew the steam through the sparge tube into the hot oil, providing sparge steam. After 30 minutes the vessel was removed from the heat source. After the oil had cooled to below 80° C., the vacuum was broken with nitrogen gas.

To investigate the effects of alkali refining (chemical refining) of palm oil, which is not normally carried out with palm oil, a second sample of bleached palm oil containing 0.8 ppm glycidyl esters was subjected to alkali refining as follows: 600 grams of refined, bleached (RB) palm oil containing 5.9% free fatty acids was heated to 40° C. and stirred with 29 mL of a 20% solution of sodium hydroxide at 200 RPM stirring for 30 minutes at 40° C. The mixture was heated to 65° C. and stirred at 65 ° C. with 110 RPM mixing for 10 minutes. The heated mixture was centrifuged for 10 minutes at 3000 RPM, then heated and stirred at 80° C. for 15 minutes. Heated water (100 mL, 80° C.) was added and the mixture was stirred at 300 RPM for one hour. The mixture was centrifuged and the palm oil layer was recovered and dried under vacuum at 90° C. and physically refined (Table 1A). In another experiment, the alkali refined bleached palm oil was contacted with TriSyl™ adsorbent as outlined below and subjected to physical refining. A third sample of bleached palm oil containing 0.8 ppm glycidyl esters was contacted with TriSyl 500™ (W. R. Grace, Columbia, Md.) silica adsorbent as follows: bleached palm oil was heated to 70° C. and TriSyl™ silica (3 weight percent) was added to the oil; the slurry was mixed for ten minutes. The slurry was heated to 90° C. under vacuum (125 mm Hg) for 20 minutes for drying prior to removing the adsorbent by filtration through #40 filter paper. The adsorbent-treated oil was physically refined at 260° C. for 30 minutes with 3% steam and 3 mm Hg vacuum.

TABLE 1A
Removal of glycidyl esters from bleached physically
refined palm oil by contact with an adsorbent.

| | GE in oil after physical refining |
|---|---|
| Oil + treatment | (ppm) |
| Starting palm oil | 0.8 |
| Physically refined palm oil | 15.6 |
| Alkali refined palm oil + | 31.8 |
| physical refining | |
| Alkali refined palm oil + | 24.3 |
| contacting with TriSyl + | |
| physical refining | |
| Starting bleached palm oil + | nd |
| contacting with TriSyl + | |
| physical refining | |

GE = glycidyl esters.
nd = not detected.
Limit of detection: 0.1 ppm GE.

Physical refining of palm oil in the control experiment caused an undesirable increase in the content of glycidyl esters in palm oil. Starting palm oil contained 0.8 ppm glycidyl esters, but when it was subjected to physical refining, the content of glycidyl esters in the palm oil increased from 0.8 ppm glycidyl esters to 15.6 ppm.

When palm oil that was alkali refined in the next experiment was then physically refined, the content of glycidyl esters undesirably increased even more, from 0.8 ppm to 31.8 ppm.

When palm oil was alkali refined, then contacted with TriSyl™ adsorbent, and then physically refined, the content of glycidyl esters did not increase as much but was still undesirably high, as it increased from 0.8 ppm to 24.3 ppm.

However, when palm oil was contacted with TriSyl™ adsorbent, then physically refined, the glycidyl esters decreased from the initial 0.8 pm to less than 0.1 ppm glycidyl esters.

### Example 1B

Bleached palm olein (ADM, Quincy, Ill.) containing 35.0 ppm glycidyl esters was incubated with 5 wt % Novozymes TL IM™ lipase at 70° C. for 4 hours in the absence of additional alcohol, fatty acid, or oil. Novozymes TL IM™ lipase is an immobilized enzyme, which when contacted with palm olein under these conditions catalyzed the interesterification of esters in the palm olein. After the reaction, the interesterified (lipase-contacted) palm olein was physically refined for 30 minutes at 240° C. under 3 mm Hg vacuum with 3% sparge steam (Table 1 B).

TABLE 1B
Effect of enzymatic interesterification and physical
refining on bleached palm olein.

| Reaction time (min) | GE (ppm) |
|---|---|
| 0 (starting oil) | 35.0 |
| 30 | 31.1 |
| 60 | 28.2 |
| 120 | 30.3 |
| 240 | 28.3 |
| 240 minutes, after physical refining | 8.4 |

Limit of detection: 0.1 ppm GE.

Contacting bleached palm olein with an enzyme resulted in a decrease of glycidyl esters in palm olein of about 10-20 percent (Table 1B). After physical refining of interesterified (lipase-contacted) oil at 240° C., the level of glycidol esters in lipase-contacted steam refined palm olein was reduced to about a third of the level in the palm olein before physical refining (from 35.0 ppm to 8.4 ppm)

### Example 1C

A sample of crude palm oil (ADM, Hamburg, Germany) containing 7.9% free fatty acids (FFA) and 0.2 ppm glycidyl esters was subjected to physical refining by steam distilling at 260° C. for 30 minutes with 3% steam at 3 mm vacuum. The content of glycidyl esters undesirably increased from 0.2 ppm to 15.9 ppm in the physically refined palm oil.

A second sample of the same crude palm oil was incubated with Novozymes 435™ lipase (10%) at 70° C. overnight under vacuum. Under these conditions the lipase catalyzed the esterification of free fatty acids in the palm oil. After the incubation, the content of free fatty acids had decreased from 7.9% to 1.9% and the content of glycidyl esters in the oil had decreased from 0.2 ppm to less than 0.1 ppm. The incubated oil was subjected to physical refining by steam distillation at 260° C. for 30 minutes with 3% steam at 3 mm vacuum to yield a lipase-contacted (esterified) steam distilled oil containing 0.9% free fatty acids and only 0.9 ppm glycidyl esters. Limit of detection: 0.1 ppm GE.

### Example 1D

Bleached palm olein (ADM, Quincy Ill.) containing 16.4 ppm glycidyl esters was subjected to rebleaching with 0.2% or 0.4% SF105™ bleaching clay at 110° C. for 30 minutes under 125 mm Hg vacuum as follows: palm olein was heated while being agitated with a paddle stirrer at 400-500 rpm until the oil temperature reached 70° C. Bleaching clay (SF105™, 0.2% or 0.4% by weight, Engelhard BASF, N.J.) was added to the oil and agitation continued at 70° C. for 5 minutes. Vacuum (max. 5 torr) was applied and the mixture was heated to 110° C. at rate of 2-5° C./min. After reaching 110° C., stirring and vacuum were continued for 20 minutes. After 20 minutes, agitation was stopped and the heat source was removed. After allowing the activated bleaching clay to settle for 5 minutes, the oil temperature had cooled to less than 100° C. Vacuum was released and a sample of oil was vacuum filtered using Buchner funnel and Whatman #2 filter paper.

Duplicate experiments were carried out, and the second example of each set was subjected to low-temperature, short time deodorization substantially as described for physical refining in 1A, except the temperature was low and the duration was short (200 ° C., 3% steam, 3 mm Hg vacuum for 5 minutes, Table 1D).

TABLE 1D
Effect of rebleaching palm olein with SF105 ™ bleaching
clay with and without low temperature, short-time deodorization.

| Rebleaching clay dosage (%) | Condition | GE (ppm) |
|---|---|---|
| Bleached palm olein starting material | — | 16.4 |
| 0.2% | Undeodorized | 5.7 |
| 0.2% | Deodorized | 5.5 |
| 0.4% | Undeodorized | nd |
| 0.4% | Deodorized | 0.2 |

nd = not detected.
Limit of detection: 0.1 ppm GE.

Rebleaching palm olein with 0.2% SF105™ reduced the content of glycidyl esters to about a third of the original level. After deodorizing the rebleached palm olein at 200° C. for five minutes, the glycidyl ester content of the oil had not increased. Rebleaching palm olein with 0.4% BASF SF105™ reduced the content of glycidyl esters to undetectable. After low-temperature deodorization (200° C. for 5 minutes), the glycidyl ester content of the oil had increased slightly to 0.2 ppm.

### Example 1E

Deodorized palm oil (ADM, Hamburg, Germany) containing 18.8 ppm glycidol esters was redeodorized in the laboratory substantially as described in Example 1D.

In order to determine whether treatment of bleached palm oil before deodorizing would affect formation of glycidyl esters in deodorization, deodorized palm oil was contacted with adsorbents and redeodorized (Table 1E). Deodorized palm oil was incubated with the adsorbents at 70° C. for 30 min under 125 mm Hg vacuum. Adsorbents included magnesium silicate (Magnesol R60™, Dallas Group, Whitehouse, N.J.), silica gel (Fisher Scientific No. S736-1), acidic alumina (Fisher Scientific No. A948-500), and acid washed activated carbon (ADP™ carbon, Calgon Corp., Pittsburg, Pa.).

TABLE 1E
Effect of contacting deodorized palm oil containing 18.8 ppm
glycidyl esters with adsorbents on development of glycidyl
esters (GE) in subsequent redeodorization.

| | GE (ppm) after |
|---|---|
| | treatment & |
| Treatment | redeodorization |
| 10% Magnesol R60 ™ | 35.1 |
| 10% silica gel | 16.9 |
| 10% acidic alumina | 21.4 |
| 5% ADP carbon | 22.2 |

Limit of detection: 0.1 ppm GE.

Contacting oil with Magnesol,™ carbon, or alumina before redeodorizing the deodorized palm oil caused an increase in glycidol esters. Contacting oil with silica gel before redeodorizing the oil caused a very slight decrease in the levels of glycidyl esters formed.

### Example 2A

Refined, bleached soybean oil (“RB soy”) (ADM, Decatur, Ill.) without detectable glycidyl esters and bleached palm oil (ADM, Hamburg, Germany) containing 0.1 ppm glycidyl esters were each steam distilled with 3% sparge steam under 3 mm Hg vacuum for 30 minutes at variable temperatures substantially as in Example 1A and as outlined in Table 2A.

TABLE 2A
Effect of deodorization of RB soybean oil and bleached palm
oil on glycidol esters (GE) at various temperatures.

| Oil, Deodorization Temperature (° C.) | GE (ppm) |
|---|---|
| RB soy control | nd |
| RBD soy, 230 | nd |
| RBD soy, 240 | 1.3 |
| RBD soy, 300 | 13.6 |
| Bleached palm control | nd |
| Bleached deodorized palm, 230 | 1.5 |
| Bleached deodorized palm, 240 | 2 |

RBD = refined, bleached, deodorized.
nd = not detected.
Limit of detection: 0.1 ppm GE.

Deodorization at 230° C. resulted in RBD soy oil that had less than 0.1 ppm glycidyl esters (Table 2A). Glycidyl esters were formed in soybean oil sparged with water steam during deodorization at 240° C. and greater levels were formed during deodorization at 300° C. Unlike soybean oil deodorized at 230° C., in bleached palm oil deodorized at 230° C., the level of glycidyl esters increased. Glycidyl esters increased to even higher levels in bleached palm oil deodorized at 240° C.

### Example 2B

Refined, bleached soybean oil (ADM, Decatur, Ill.) without detectable glycidyl esters or bleached palm oil (ADM, Hamburg, Germany) without detectable glycidyl esters were lab deodorized (soybean oil) or physically refined (palm oil) under 3 mm Hg vacuum for 30 minutes substantially as in Example 1 and as outlined in Table 28. In one test, 35 ppm SF105™ bleaching clay was added to soybean oil before deodorizing with 3% water steam. In two tests, RB soybean oil was deodorized with 95% ethanol sparge prepared by diluting absolute ethanol (Sigma-Aldrich) to 95% with water (9% and 10.8% of oil volume) wherein the ethanol sparge replaced conventional water (steam) sparge. In two tests, water (steam) sparge was replaced with gas sparge (nitrogen or carbon dioxide).

TABLE 2B
Deodorization tests with unconventional deodorization/physical
refining sparge compositions.

| | Deodorization/Physical refining | |
|---|---|---|
| Oil, Temperature | condition | GE (ppm) |
| RB soy (starting oil) | — | nd |
| RBD soy, 240° C. | Bleaching clay (35 ppm) | 1.3 |
| RBD soy, 220° C. | Ethanol sparge, 9% | nd |
| RBD soy, 240° C. | Ethanol sparge, 10.8% | nd |
| Bleached palm (starting oil) | — | 0.1 |
| Bleached palm, 260° C. | 3% water sparge (control) | 15.3 |
| Bleached palm, 260° C. | Nitrogen sparge | 9.8 |
| Bleached palm, 260° C. | Carbon dioxide sparge | 9.4 |

nd = not detected.
Limit of detection: 0.1 ppm GE.

Glycidyl esters were formed in deodorization at 240° C. when bleaching clay was added to the RB soy oil in the deodorization vessel. However, replacing water steam sparging with ethanol resulted in deodorized oil in which glycidyl esters were removed, even at 240° C. When bleached palm oil was physically refined at 260° C., the GE content was 15.3 ppm. Replacing conventional water with nitrogen or carbon dioxide in physical refining of bleached palm oil resulted in lower levels of glycidyl esters. The rate of sparge of the gases was difficult to measure and control in this test. Deodorizing soy oil with ethanol sparge resulted in a composition comprising a refined, bleached, deodorized soybean oil containing less than 0.1 ppm glycidyl esters. Steam refining bleached palm oil with a carbon dioxide sparge or nitrogen sparge resulted in a composition comprising a bleached physically refined palm oil having a lower content of glycidyl esters than the same bleached palm oil refined by physical refining.

### Example 3A

Refined, bleached, deodorized (RBD) corn oil (ADM, Decatur, Ill.) containing 2.2 ppm glycidyl esters was contacted with solutions of acid as outlined in Table 3A. Acid solution (1 part) was contacted with corn oil (1000 parts) by shear mixing for period outlined in Table 3B. The mixture was then stirred for 30 minutes and washed repeatedly with water until the pH of the wash water was neutral after washing.

TABLE 3A
Effect of contacting RBD corn oil with acid solutions and
shear mixing on glycidyl ester (GE) content.

| | Shear mix | |
|---|---|---|
| Acid | time (min) | GE (ppm) |
| Untreated RBD corn oil | — | 2.2 |
| 50% Citric acid | 2 min | 1.9 |
| 50% Citric acid | 4 min | 2.2 |
| 50% Citric acid | 8 min | 2.7 |
| 50% Malic acid | 4 min | 2.1 |
| 85% Phosphoric Acid | 4 min | 0.3 |
| 85% Lactic acid | 4 min | 2.2 |
| 30% Ascorbic acid | 4 min | 2.5 |
| 50% EDTA | 4 min | 2.0 |
| 50% Succinic acid | 4 min | 2.4 |

Limit of detection: 0.1 ppm GE.

Contacting RBD corn oil with organic acid solutions or EDTA solution exerted little or no reduction in glycidyl esters. Contacting RBD corn oil with 85% phosphoric acid solution and shear mixing for 4 minutes reduced the content of glycidyl esters and produced RBD corn oil containing 0.3 ppm glycidyl esters.

### Example 3B

Refined, bleached deodorized soybean oil (ADM, Decatur, Ill.) without detectable glycidyl esters was spiked with glycidyl stearate to yield RBD soybean oil containing 13.6 ppm glycidyl stearate. The spiked RBD oil was subjected to treatment with acid solutions substantially as outlined in Example 3A and Table 3B. Spiked RBD oil was also contacted with magnesium silicate (Magnesol R60™, Dallas Group, Whitehouse, N.J.; 1% of oil, 150° C., 5 minutes).

TABLE 3B
Effect of contacting glycidyl ester-spiked RBD soybean oil with
acid solutions or Magnesol R60 ™ on levels of glycidyl esters.

| | Glycidyl esters (ppm) |
|---|---|
| Starting spiked RBD soybean oil | 13.6 |
| Citric acid 0.1% | 14.5 |
| Citric acid 0.2% | 15 |
| Phosphoric acid 0.1% | 7.9 |
| Magnesol R60 ™ (1%, 150 C, 5 min) | nd |

nd = not detected.
Limit of detection: 0.1 ppm GE.

Treatment of oil with citric acid solutions increased the level of glycidyl esters in the RBD oil. Phosphoric acid treatment caused a reduction in glycidyl esters in RBD soybean oil. Only treatment with Magnesol R60TM reduced glycidyl esters to less than 0.1 ppm.

### Example 4A

Refined, bleached, deodorized soybean oil (ADM, Decatur, Ill.) containing 0.02% free fatty acids (FFA) without detectable glycidyl esters was spiked with glycidyl stearate to yield RBD soybean oil containing 11.1 ppm glycidyl stearate. The spiked RBD soybean oil was subjected to rebleaching for 30 minutes at 125 mm Hg vacuum with beaching clays, dosages and times listed in Table 4A1 substantially as described in Example 1D. Subsequently, re-bleached oil was tested for glycidyl esters and the color was evaluated substantially according to A.O.C.S method Cg 13b-45 (Table 4A1). The spiked RBD soybean oil had good color (0.5 R and 4.5 Y) before rebleaching.

TABLE 4A1
Rebleaching conditions of RBD soybean oil spiked to
contain 11.1 ppm glycidyl esters, and levels of glycidyl
esters and color after rebleaching.

| | | | Re- | | |
|---|---|---|---|---|---|
| | Bleaching | Bleaching | bleaching | GE in Re- | |
| | Clay | Clay Dosage | Temp | bleached Oils | Re-bleached |
| # | Type | (%) | (° C.) | (ppm) | Color (R; Y) |
| | None | | | 11.1 | 0.5; 4.5 |
| | (control) | | | | |
| 1 | SF105 ™ | 0.1 | 70 | 8.4 | 0.4; 3.8 |
| 2 | SF105 ™ | 0.4 | 70 | 2.0 | 0.4; 4.0 |
| 3 | SF105 ™ | 0.1 | 110 | 3.9 | 0.5; 4.2 |
| 4 | SF105 ™ | 0.2 | 110 | nd | 0.4; 4.0 |
| 5 | SF105 ™ | 0.4 | 110 | nd | 0.3; 3.6 |
| 6 | BioSil ™ | 0.2 | 110 | nd | 0.5; 6.3 |
| 7 | BioSil ™ | 0.4 | 110 | nd | 0.5; 4.5 |
| 8 | Tonsil | 0.4 | 110 | nd | 0.6; 4.9 |
| | 126FF ™ | | | | |

SF105 ™ and Tonsil 126FF ™ are acid-activated bleaching clays.
nd = not detected.
Limit of detection: 0.1 ppm GE.

Dose-dependent and temperature-dependent effects on glycidyl ester removal in rebleaching were observed. Rebleaching at 70° C. with SF105™ bleaching clay at 0.1% and 0.4%, and at 110° C. with SF105™ bleaching clay used at 0.1%, caused a reduction but not elimination of glycidyl esters. When the level of SF105™ bleaching clay was increased to 0.2% and 0.4% at 110° C., glycidyl esters were removed from the oil to yield rebleached oil without detectable glycidyl esters. Bleaching with Biosil™ and Tonsil™ 126 FF at 110° C. at the levels tested also resulted in oils having less than 0.1 ppm glycidyl esters. The level of free fatty acids in RBD oil and all rebleached RBD oil samples was unchanged at 0.02%. Rebleaching RBD oil containing 11.1 ppm glycidyl esters removed some or all of the glycidyl esters and gave oils with good color; however, the flavors and odors of all rebleached oils were objectionable.

Rebleached oils without detectable glycidyl esters but having objectionable odor and flavor from Table 4A1 were subjected to low temperature, short time deodorization after rebleaching substantially as outlined in Example 1 under conditions outlined in Table 4A2. Rebleached, redeodorized oil was tested for glycidyl esters and the flavor was evaluated substantially according to A.O.C.S method Cg 2-83.

TABLE 4A2
Low-temperature, short time redeodorization of rebleached
RBD soybean oil from Table 4A1.

| | | | | | GE in |
|---|---|---|---|---|---|
| | | | | | finished |
| | | | | | RBD |
| | Deodorization | Deodorization | Steam | Flavor after | oils |
| # | temp (° C.) | time (min) | rate (%) | deodorization | (ppm) |
| 1 | 210 | 10 | 2 | Good, Pass | nd |
| 2 | 210 | 5 | 0.7 | Good, Pass | nd |
| 6 | 200 | 5 | 1.3 | Good, Pass | nd |
| 7 | 180 | 5 | 1.1 | Good, Pass | nd |
| 8 | 180 | 5 | 1.5 | Good, Pass | nd |

Numbers in first column refer to Table 4A1.
nd = not detected.
Limit of detection: 0.1 ppm GE.

Glycidyl esters were not detected in any RBD soybean oil samples that had been rebleached and deodorized at low temperature and for short time after rebleaching (Table 4A2).

Re-bleaching spiked soybean oil containing 11.1 ppm glycidyl esters was effective in producing an oil without detectable glycidyl esters, and deodorizing at low temperatures (180-210° C.) for short times (5-10 minutes) after rebleaching was effective in removing objectionable flavors from the re-bleaching treatment with no increase in glycidyl esters. Oil having good flavor without detectable glycidyl esters was obtained by rebleaching, followed by low temperature, short time redeodorizing.

### Example 4B

Palm stearin (ADM, Quincy, Ill.) with Lovibond color values of 3.8 red and 26 yellow contained 11.3 ppm glycidyl esters (GE). The palm stearin had high free fatty acids (0.30% FFA) even though the source palm oil had been bleached and steam distilled in the country of origin before fractionation and transport.

Palm stearin was treated by rebleaching and low temperature, short-time deodorization. The palm stearin was rebleached with BASF SF105™ bleaching clay at different levels, temperatures, and times as outlined in Table 4B1. The levels of glycidyl esters in the re-bleached oils were determined and the re-bleached oils were deodorized at low temperatures for short times (Table 4B1). In a control experiment, rebleached oil was subjected to physical refining at 260° C. for 30 minutes (Table 4B2), resulting in a significant increase in glycidyl esters.

TABLE 4B1
Re-bleaching and deodorizing of palm stearin containing 11.3 ppm glycidyl esters.

| SF105 ™ | Re- | Re- | GE in | Deod | Deod | | | GE in |
|---|---|---|---|---|---|---|---|---|
| dose | bleach | bleach | rebleached | temp | time | FFA | Color | deod. oil |
| (%) | temp (° C.) | time (min) | oil (ppm) | (° C.) | (min) | (%) | (R; Y) | (ppm) |
| 0.2 | 110 | 30 | 4.6 | 180 | 10 | 0.28 | 2.4/19 | Not |
| | | | | | | | | tested |
| 0.4 | 110 | 30 | 2.6 | 200 | 10 | 0.29 | 2.4/19 | 2.8 |
| 0.6 | 110 | 30 | 0.4 | 200 | 10 | 0.29 | 3.3/22 | 0.4 |
| 0.4 | 150 | 15 | nd | 180 | 10 | 0.29 | 3.2/20 | nd |
| 0.4 | 150 | 5 | 2.7 | 180 | 10 | 0.28 | 2.4/19 | 4.5 |

nd = not detected. Limit of detection: 0.1 ppm GE.

TABLE 4B2
Results of rebleaching and physical refining of
palm stearin containing 11.3 ppm glycidyl esters.

| SF105 ™ | Re- | Re- | GE in | P.R. | P.R. | | | GE in |
|---|---|---|---|---|---|---|---|---|
| dose | bleach | bleach | rebleached | temp | time | FFA | Color | P.R. oil |
| (%) | temp (° C.) | time (min) | oil (ppm) | (° C.) | (min) | (%) | (R; Y) | (ppm) |
| 0.4 | 150 | 30 | nd | 260 | 30 | 0.06 | 3.8/30 | nd |

P.R. = Physical refining

All of the rebleached and deodorized or physically refined palm stearin samples passed the flavor screen. Re-bleaching palm stearin followed by low-temperature deodorization was effective in removing glycidyl esters from palm stearin. However, low-temperature deodorization was not able to reduce the FFA in RBD palm stearin to a satisfactory level.

### Example 4C

Palm olein (ADM, Quincy, Ill.) having Lovibond color values of 3.2 red and 38 yellow and 40.1 ppm glycidyl esters was treated by rebleaching and deodorizing or physical refining. The incoming palm olein had high free fatty acids (0.16% FFA) even though the source palm oil had been bleached and physically refined in the country of origin before fractionation and transport.

Palm olein was rebleached with BASF SF105™ bleaching clay at different clay levels, temperatures, and times (Table 4C1). The levels of glycidyl esters in the rebleached palm oleins were determined and the rebleached palm oleins were then deodorized at low temperature for various times (Table 4C1). For comparison, palm olein was rebleached and physically refined (Table 4C2).

TABLE 4C1
Re-bleaching and deodorizing of palm olein containing 40.1 ppm glycidyl esters.

| SF105 ™ | Re- | Re- | GE in | Deod | Deod | | | GE in |
|---|---|---|---|---|---|---|---|---|
| dose | bleach | bleach | rebleached | temp | time | FFA | Color | deod. oil |
| (%) | temp (° C.) | time (min) | oil (ppm) | (° C.) | (min) | (%) | (R; Y) | (ppm) |
| 0.4 | 150 | 5 | 9 | 180 | 10 | 0.18 | 3.4/34 | 10.5 |
| 0.4 | 110 | 30 | nd | 200 | 10 | 0.13 | 3.5/38 | 5.5 |
| 0.4 | 110 | 30 | nd | 180 | 10 | 0.16 | 3.3/32 | 8.6 |
| 0.6 | 110 | 30 | nd | 200 | 10 | 0.14 | 43.4/32 | nd |

TABLE 4C2
Rebleaching and physical refining of palm
olein containing 40.1 ppm glycidyl esters.

| SF105 ™ | Re- | Re- | GE in | P.R. | P.R. | | | GE in |
|---|---|---|---|---|---|---|---|---|
| dose | bleach | bleach | rebleached | temp | time | FFA | Color | P.R. oil |
| (%) | temp (° C.) | time (min) | oil (ppm) | (° C.) | (min) | (%) | (R; Y) | (ppm) |
| 0.4 | 150 | 15 | nd | 260 | 30 | 0.05 | 3.8/34 | 42 |
| 0.4 | 150 | 30 | 2.3 | 200 | 10 | | | 1.5 |
| 0.4 | 150 | 30 | 1.5 | 200 | 10 | | | 1.6 |

P.R. = Physical refining nd = not detected. Limit of detection: 0.1 ppm GE.

All of the rebleached oils had good color and passed the flavor test after rebleaching and deodorizing or physical refining. This method of rebleaching palm olein and deodorizing the palm olein at low temperature and for short times after rebleaching resulted in a composition comprising deodorized palm olein having a lower level of glycidyl esters than the starting (physically refined) palm olein.

### Example 5A

Bleached palm oil (ADM, Hamburg, Germany, 600 grams) was contacted with Novozymes TL IM™ lipase (60 grams, 10%) at 70° C. for two hours in an interesterification reaction to produce interesterified oil. Some of the interesterified oil (200 grams) was subjected to physical refining by steam distillation at 260° C. for 30 minutes with 3% steam at 3 mm vacuum substantially as in example 1A to yield a physically refined lipase-contacted (interesterified) oil. Some of the interesterified oil (250 grams) was subjected to rebleaching by contacting it with SF105™ bleaching clay (2%) substantially as described in example 1D, then subjected to physical refining by steam distillation at 260° C. for 30 minutes with 3% steam at 3 mm vacuum substantially as in example 1A to yield a rebleached physically refined lipase-contacted (interesterified) oil. The content of glycidyl esters in samples taken after various processing steps was determined Table 5A).

TABLE 5A
Lipase-contacting and further processing of palm oil.

| Oil description | GE in oil (ppm) |
|---|---|
| Starting palm oil | 15.9 |
| Lipase-contacted oil | 17.2 |
| Lipase-contacted oil, after physical refining | 48.7 |
| Lipase-contacted oil, after bleaching | 7.3 |
| Lipase-contacted oil, after bleaching and physical | 38.4 |
| refining | |

The starting palm oil contained 15.9 ppm glycidyl esters. After contacting with a lipase the glycidyl ester content had hardly changed. On physical refining of the interesterified oil, the content of glycidyl esters increased dramatically. In spite of the teaching in the art that bleaching interesterified oil is not necessary, bleaching the lipase-contacted oil decreased the content of glycidyl esters from 15.9 ppm to 7.3 ppm. The additional step provided oil of higher quality than when no additional step was applied. Subsequent physical refining caused an increase in glycidyl esters.

It is widely taught in the art of oil interesterification that the use of enzymes to catalyzed interesterification obviates the need for bleaching because the products of interesterification by contacting oils with a lipase are much more pure than the products of chemical processes. Thus, purification steps are avoided. As reported in the Oil Mill Gazetteer (Vol. 109, June 2004), “With a chemical system, a reactor is also needed, but much higher temperatures are required than with enzymes. Because a dark color develops during the chemical process, extensive purification of the oil is needed, This is not the case if enzymes are used.” As reported in Palm Oil Developments (39 p 7-10, http://palmoilis.mpob.gov.my/publications/pod39-p7.pdf; accessed Oct. 30, 2009); “With enzymatic interesterification, the process is gentler, does not darken the oil, and eliminates the expensive post-bleaching operation.” The elimination of bleaching steps using lipase interesterification to produce edible fats is widely recognized: “The enzymatic process is much simpler than the chemical and there is no requirement for any post-treatment of the interesterified oil afterwards.” As reported in BioTimes (December 2006, Novozymes BV, Bagsvaerd, Denmark, publisher) “The main advantages of the enzymatic process are a mild temperature, no neutralisation or bleaching is needed, no liquid effluents are generated, and the enzymes are safer to handle than very reactive and unstable chemicals.”

However, in spite of this teaching, we found that bleaching lipase-contacted oil decreased the content of glycidyl esters.

### Example 5B

Refined, bleached soybean oil (80 parts) was blended with fully hydrogenated soybean oil (20 parts, ADM, Decatur, Ill.) and enzymatically interesterified by contacting with TL IM™ lipase (5%) for 4 hours substantially as described in example 1B to produce enzymatically interesterified oil. The RB soybean oil, the fully hydrogenated soybean oil, and the enzymatically interesterified oil did not contain detectable levels of glycidyl esters (Limit of detection: 0.1 ppm GE). The enzymatically interesterified oil was subjected to physical refining at 260° C. substantially as outlined in Example 1A to yield an interesterified oil containing 4.6 ppm glycidyl esters. When the enzymatically interesterified oil was subjected to physical refining at 240° C. the interesterified soybean oil contained 0.3 ppm glycidol esters.

### Example 6

Refined, bleached soybean oil (80 parts) was blended with fully hydrogenated soybean oil (20 parts, ADM, Decatur, Ill.) and subjected to chemical interesterification substantially as follows: the oil mixture (600 grams) was dried by heating for 20 min under vacuum and stirring at 90° C. After drying, the oil was cooled to 85° C., blended with 2.1 grams (0.35) % sodium methoxide (Sigma Aldrich) and stirred for 1 hour under vacuum at 85° C. to produce chemically interesterified oil. Wash water (48 mL) was added to inactivate the catalyst and stop the reaction and agitated at 200 RPM for 15 minutes. The agitation was stopped and the oil was allowed to incubate for 5 minutes before decanting the oil. The oil was washed twice more with water in the same way. The oil was dried by incubating it at 90° C. Some of the chemically interesterified oil (200 grams) was deodorized at 240° C. for 30 minutes substantially as outlined in Example 1A to provide deodorized chemically interesterified oil. Some of the chemically interesterified oil (200 grams) was rebleached substantially as outlined in Example 1D with 1.5% SF105 clay for 30 minutes at 110° C. under 125 mm Hg vacuum to provide rebleached chemically interesterified oil. The rebleached chemically interesterified oil was deodorized substantially as outlined in Example 1A to provide deodorized rebleached chemically interesterified oil (Table 6).

TABLE 6
Chemical interesterification and further processing of soybean oil.

| | GE (ppm) |
|---|---|
| Feed for CIE | nd |
| Reaction mixture after CIE | 373.8 |
| CIE deodorized without bleaching | 198.2 |
| Bleached CIE | nd |
| Bleached and deodorized CIE | 12.1 |

After chemical interesterification, the level of glycidyl esters in the oil increased substantially. The level of glycidyl esters in deodorized chemically interesterified oil was reduced substantially to about half the level of glycidyl esters in the chemically interesterified oil. The level of glycidyl esters in bleached chemically interesterified oil was reduced to below detectable levels. The level of glycidyl esters in deodorized rebleached chemically interesterified oil increased to 12.1 ppm glycidyl esters.

### Example 7A

Glycidyl stearate was blended into refined, bleached, deodorized soybean oil (ADM, Decatur Ill.) to obtain a spiked oil containing 513 ppm glycidyl esters. 3-Monochloropropanediol monoesters or diesters were not detected in the oil (<0.1 ppm). A ten gram sample of the starting oil was removed as a control and tested to determine the content of glycidyl esters and monoglycerides. The remaining oil was rebleached using 5 wt % SF105™ bleaching clay at 150° C. under 125 mm Hg vacuum for 30 minutes as follows: oil was heated while being agitated with a paddle stirrer at 400-500 rpm until the oil temperature reached 70° C. Bleaching clay (SF105™, Engelhard BASF, N.J., 5% by weight of oil) was added to the oil and agitation continued at 70° C. for 5 minutes. Vacuum (125 torr) was applied and the mixture was heated to 150° C. at rate of 2-5° C./min. After reaching 150° C., stirring and vacuum were continued for 20 minutes. After 20 minutes, agitation was stopped and the heat source was removed. After allowing the activated bleaching clay to settle for 5 minutes, the oil temperature had cooled to less than 100° C. Vacuum was released and the bleached oil was vacuum filtered using Buchner funnel and Whatman #40 filter paper. The rebleached oil was weighed.

Spent filter clay was recovered from the filter paper and extracted with 100 ml hexane for one hour with occasional stirring. The slurry was filtered and the clay was extracted with 100 ml chloroform for one hour with occasional stirring. The slurry was filtered and the clay was extracted with 100 ml methanol for one hour with occasional stirring, then the slurry was filtered and the clay was extracted with 100 ml methanol for one hour with occasional stirring for a second time. After the extraction solutions were combined and the solvent was evaporated, 5.58 grams of oil extracted from the clay were recovered.

TABLE 7A
Content of glycidyl esters and stearate
monoacylglycerol (monostearin).

| | | | | Monostearin |
|---|---|---|---|---|
| | Glycidyl esters | Monostearin | Quantity | recovered |
| | (ppm) | (ppm) | (grams) | (mg) |
| Starting oil | 513 | <1 | 350 | 189 |
| Rebleached oil | nd | 147 | 332.6 | 49 |
| Oil from clay | nd | 5617 | 7.1 | 40 |

nd = not detected.
Limit of detection: 0.2 ppm GE.

The glycidyl esters were reduced to below detection levels in the rebleached oil, and no glycidyl esters were extracted from the spent clay. While the absence of glycidyl esters after rebleaching may have been due to irreversible adsorption to the bleaching clay, the simultaneous appearance of monostearin indicates that the GE were probably converted to monostearin in rebleaching. About half (47 mole percent) of the glycidyl stearate was recovered in the form of monostearin.

### Example 7B

A second spiked oil was prepared and bleached substantially as in Example 7A to obtain a spiked RBD soybean oil containing 506 ppm glycidyl esters. 3-Monochloropropanediol was not detected in the oil (<0.1 ppm). The spiked oil (300 grams) was rebleached substantially as in Example 6A except that after the oil was heated to 70° C., 1.5 ml (0.5% based on the oil) deionized water was added to the oil, with vigorous agitation (475 rpm) for 5 minutes. Then, bleaching clay (SF105™, 15 grams, 5%) was added and the slurry was mixed for 5 minutes. The slurry was heated to 90° C. without vacuum and held for 20 minutes. Then, vacuum was applied to the slurry and it was heated to 110° C. and held at 110° C. for 20 minutes. The rebleached oil was cooled and filtered through #40 filter paper. Rebleached oil 284.4 grams) was recovered and the content of monostearin was determined. The spent clay was extracted substantially as in Example 7A and 6.88 grams of oil was recovered from the bleaching clay.

TABLE 7B
Content of glycidyl esters and monostearin in rebleached oil
and bleaching clay after bleaching with 0.5% added water.

| | | | | Monostearin |
|---|---|---|---|---|
| | Glycidyl esters | Monostearin | Quantity | recovered |
| | (ppm) | (ppm) | (grams) | (mg) |
| Starting oil | 506 | <1 | 300 | 155.20 |
| Rebleached oil | nd | 19 | 284.4 | 5.40 |
| Oil from clay | 1.1 | 18279 | 6.88 | 125.76 |

nd = not detected.
Limit of detection: 0.2 ppm GE.

The content of glycidyl esters in the oil was reduced from 506 ppm to below detection limits by mixing water into the oil, then rebleaching. Monostearin was recovered from bleaching clay, and the RBD soybean oil that was substantially free from monostearin before rebleaching contained significant quantities after rebleaching after 0.5% water was mixed into the oil. The simultaneous appearance of monostearin indicates that the GE were converted to monostearin by rebleaching in the presence of added water. In addition, no MCPD monoesters or MCPD diesters were detected in the rebleached oil or the oil extracted from bleaching clay. A large amount (85 mole percent) of the glycidyl stearate was recovered in the form of monostearin.

### Example 7C

A third spiked oil was prepared and bleached substantially as in Example 7A to obtain a spiked RBD soybean oil containing 72.6 ppm glycidyl esters. 3-Monochloropropanediol esters were not detected in the oil (<0.1 ppm). Rebleaching with varied amounts of water added (none, 0.25%, 0.5% or 1.0%, based on oil) was carried out on 300 gram lots of spiked oil substantially as outlined in Example 7B, except that only 2 wt% bleaching clay was added. Oil was recovered from each spent bleaching clay substantially as outlined in Example 7A.

TABLE 7C
Content of glycidyl esters and monostearin.

| | Glycidyl | Monostearin | | Total monostearin | |
|---|---|---|---|---|---|
| | esters | obtained | Quantity | recovered | |
| | (ppm) | (mg) | (grams) | (mg) | (%) |
| Starting oil | 95.3 | <1 | 300 | — | — |

No water addition

| | | | | | |
|---|---|---|---|---|---|
| Rebleached oil | nd | <1 | 284 | 8.3 | 36 |
| Oil from clay | not tested | 8.3 | 2.28 | | |

0.25% water addition

| | | | | | |
|---|---|---|---|---|---|
| Rebleached oil | nd | 10.04 | 287 | 14.94 | 65 |
| Oil from clay | not tested | 4.9 | 2.14 | | |

0.5% water addition

| | | | | | |
|---|---|---|---|---|---|
| Rebleached oil | nd | 10.44 | 290 | 20.75 | 90 |
| Oil from clay | not tested | 10.31 | 4.47 | | |

1.0% water addition

| | | | | | |
|---|---|---|---|---|---|
| Rebleached oil | nd | 10.69 | 289 | 17.46 | 76 |
| Oil from clay | not tested | 6.77 | 2.96 | | |

The starting oil contained 21.87 mg of glycidyl stearate, which is equivalent to about 23.0 mg monostearin on a molar basis.
nd = not detected.
Limit of detection: 0.2 ppm GE.

Monostearin was recovered from bleaching clay after bleaching in either the absence or the presence of added water. RBD soybean oil that was substantially free from monostearin before rebleaching was also substantially free from monostearin after bleaching without added water, but contained about 10 grams after rebleaching in the presence of 0.25% - 1.0% added water. Adding water to the oil before bleaching aided in the recovery of GE as monostearin in the rebleached oil.

1-29. (canceled)

30. A composition comprising steam refined oil wherein the level of glycidyl esters is less than or equal to 1 ppm.

31. The composition of claim 30, wherein the steam refined oil comprises at least one oil selected from the group consisting of palm oil, palm fraction, palm olein, palm stearin, corn oil, soybean oil, esterified oil, interesterified oil, chemically interesterified oil, and lipase-contacted oil.

32. A method of making the composition of claim 30 comprising contacting the steam refined oil with an adsorbent.

33. The method of claim 32, further comprising subsequently steam refining the steam refined oil.

34. The method of claim 33 wherein steam refining the steam refined oil comprises at least one of deodorization and physical refining.

35. The method of claim 32 wherein the adsorbent comprises at least one material selected from the group consisting of magnesium silicate, silica gel, and bleaching clay.

36. A method of making the composition of claim 30 comprising contacting the steam refined oil with an enzyme; and,
subsequently steam refining the oil.

37. The method of claim 36, wherein contacting the steam refined oil with an enzyme comprises at least one reaction selected from the group consisting of hydrolysis, esterification, transesterification, acidolysis, interesterification, and alcoholysis.

38. A method of making the composition of claim 32 comprising deodorizing the steam refined oil at a temperature no greater than 240 degrees C.

39. A method of making the composition of claim 32 comprising deodorizing the steam refined oil with at least one sparge selected from the group consisting of ethanol sparge, carbon dioxide sparge, and nitrogen sparge.

40. A method of making the composition of claim 33 comprising contacting the palm oil with an acid.

41. The method of claim 40, wherein the acid comprises phosphoric acid.

42. The method of claim 40, wherein contacting the steam refined oil with the acid comprises shear mixing the oil and the acid.

43. A method of making the composition of claim 30 comprising bleaching the steam refined oil, and rebleaching the steam refined oil, wherein the level of glycidol esters of the steam refined oil before rebleaching is greater than the level of glycidol esters of the steam refined oil after rebleaching the oil.

44. The method of claim 43, wherein the bleached oil includes at least one oil selected from the group consisting of refined bleached oil, refined bleached deodorized oil, and chemically interesterified oil.

45. The method of claim 43, further comprising:
mixing water into the steam refined oil; and,
rebleaching the steam refined oil.

46. The method of claim 43, further comprising deodorizing the steam refined oil subsequent to rebleaching the steam refined oil.

47. The method of claim 4, wherein the deodorizing the steam refined oil is carried out for not more than 15 minutes.

48. The method of claim 46, wherein the deodorizing the steam refined oil is carried out at a temperature not greater than 210 degrees C.

49. The composition of claim 31 further comprising physically refined palm oil having a level of glycidyl esters less than 0.1 ppm.

50. The composition of claim 31 further comprising palm olein having a level of glycidyl esters less than 0.1 ppm.

51. The composition of claim 31 further comprising physically refined palm olein having a level of glycidyl esters less than 0.3 ppm.

52. The composition of claim 30 further comprising a rebleached, redeodorized oil, the rebleached, redeodorized oil comprising:
a level of glycidyl esters less than 0.1 ppm;
a Lovibond red color value no greater than 2.0;
a Lovibond yellow color value no greater than 20.0; and
a free fatty acid content of less than 0.1%.

53. The composition of claim 52, wherein the rebleached, redeodorized oil further comprises flavor that passes when tested by the American Oil Chemists' Society method Cg-2-83.

54. The composition of claim 30 wherein the steam refined oil comprises a rebleached, steam distilled palm oil, the rebleached, steam distilled palm oil comprising:
a level of glycidyl esters below 0.2 ppm as determined by the liquid chromatography time-of-flight mass spectroscopy method;
a Lovibond red color value no greater than 3.0; and,
less than 0.1% free fatty acids.

55. The composition of claim 30 wherein the steam refined oil comprises a rebleached, steam distilled palm stearin, the rebleached, steam distilled palm stearin comprising:
a level of glycidyl esters below 0.2 ppm;
a Lovibond red color value of 4.0 or less; and
less than 0.1% free fatty acids.

56. The composition of claim 30 wherein the steam refined oil comprises a bleached lipase-contacted oil having a level of glycidyl esters less than 1.0 ppm.

57. The composition of claim 56, wherein the bleached lipase-contacted oil is deodorized.

58. A method of converting glycidyl esters into monoacylglycerols comprising:
mixing water into an oil; and,
rebleaching the oil.

59. A method of removing glycidyl esters from an oil, comprising:
at least one act selected from the group consisting of contacting the oil with an adsorbent, contacting the oil with an enzyme, deodorizing the steam refined oil with at least one sparge selected from the group consisting of ethanol sparge, carbon dioxide sparge, and nitrogen sparge, contacting the oil with an acid, rebleaching the oil, deodorizing the oil at a temperature of no greater than 240 degrees C., contacting the oil with an acid, and rebleaching the oil.
